(19) United States
(12) Patent Application Publication
(10) Pub. No.: US 2005/0049309 A1
(43) Pub. Date: Mar. 3, 2005

(54) Regulation of HIF protein levels via deubiquitination pathway

(76) Inventors: Lynn Kirkpatrick, Tucson, AZ (US); Garth Powis, Tucson, AZ (US); Sarah J. Welsh, Oxford (GB)

Correspondence Address: Pepper Hamilton LLP;One Mellon Center, 50th Floor, 500 Grant Street, Pittsburgh, PA, 15219, US

(21) Appl. No.: 10/890,756

(22) Filed: Jul. 14, 2004

Related U.S. Application Data
(60) Provisional application No. 60/487,562, filed on Jul. 14, 2003.

(51) Int. Cl. A61K 31/195

(52) U.S. Cl. 514/567

(57) ABSTRACT

The hypoxia inducible factor-1 (HIF-1) transcription factor is an important regulator of the cellular response to hypoxia. The activity of HIF-1 is regulated by the level of the HIF-1α subunit, HIF-1α, which is rapidly degraded under normoxic conditions by the ubiquitin-proteasome pathway. HIF-1α levels increase under hypoxic conditions. Many human cancers also show constitutively increased HIF-1α levels. PX-478 or S-2-amino-3-[4′-N,N,-bis(2-chloroethyl)amino]phenyl propionic acid N-oxide dihydrochloride, is a novel anticancer agent, and is capably of decreasing both constitutive and hypoxia induced HIF-1α protein levels and HIF-1 transactivation in vitro and in vivo. In method embodiments, the administration of PX-478 is independent of the pathways of HIF-1α regulation involving the von Hippel-Lindau protein and p53. PX-478 causes an increase in polyubiquitinated HIF-1α levels due to inhibition of HIF-1α deubiquitination. The levels of other proteins whose proteasomal breakdown is mediated by ubiquitination are not affected by PX-478. Deubiquitination is a novel pathway for the regulation of cellular HIF-1α levels and PX-478 is a specific inhibitor of the pathway. Therapeutic compounds for regulating cellular HIF-1α levels and methods of regulating cellular HIF-1α levels are herein provided.

PRIORITY

This application claims priority to U.S. Provisional Application No. 60/487,562, filed on Jul. 14, 2003, the contents of which are incorporated herein by reference in their entirety.

### TECHNICAL FIELD

The present invention relates to compounds, compositions, and formulations of N-oxides and derivatives thereof, particularly to N-oxides and derivatives thereof that are useful in treating diseased states, and more particularly to N-oxides and derivatives thereof that are capable of regulating HIF levels in cells under hypoxic or normoxic conditions via the ubiquitin/26S proteasome mechanism. The present invention is directed to compositions and formulas that include S-2-amino-3-[4′-N,N,-bis(2-chloroethyl)amino]phenyl propionic acid N-oxide dihydrochloride, which is also known as “PX-478” or the N-oxide of melphalan.

### BACKGROUND OF THE INVENTION

Chlorambucil derivatives have been previously described in U.S. Pat. No. 5,602,278 (“the '278 patent”), which is incorporated herein in its entirety. The '278 patent describes the use of chlorambucil and N-oxide derivates thereof in hypoxic environments, and more particularly chlorambucil in combination with hydralazine to create such reactive conditions. Additionally, in U.S. patent application Ser. No. 10/288,888, filed on Nov. 6, 2002, herein incorporated by reference, teaches the use of various compounds effective in inhibiting HIF-1α or angiogenesis under hypoxic or normoxic conditions.

Furthermore, the '278 patent describes N-oxides and derivatives of chlorambucil, specifically CHLN-O and CHL-HD, as effective alkylating agents under certain conditions (in the presence of microsomes). This reference, however, does not teach or suggest the use of all N-oxides as effective inhibitors of HIF-1α or angiogenesis.

In the '278 patent, vitro and in vivo results were described for the N-oxide derivative of chlorambucil (CHLN-O) and of the hydroxylamine derivative of chlorambucil (CHL-HD). Both compounds had a greater toxicity with reducing enzymes under hypoxia. Such biological activity was unexpected in view of the other reported results and in view of their molecular structure. Furthermore, both CHLN-O and CHL-HD were stable and produced minimal in vivo toxicity.

In the Ser. No. 10/288,888 application, the ability of CHLN-O and PX-478 to inhibit HIF-1α and to inhibit angiogenesis was more fully explained and documented.

It has been proposed that HIF is regulated via the pVHL pathway. See e.g., ,P. Maxwell, M. Wiesener, G. W. Chang, et al., “The tumor suppressor protein VHL targets hypoxia-inducible factors for oxygen-dependent proteolysis,” Nature 1999; 399: 271-275, herein incorporated by reference. Additionally, inhibition of HIF-1α has been linked to the p53 pathway. See e.g., R. Ravi, B. Mnookerjee, et al., “Regulation of tumor angiogenesis by p53-induced degradation of hypoxia-inducible factor 1 alpha,” Genes Develop 2000; 14(1):34-44, herein incorporated by reference and M. V. Blagosklonny, W. G. An, L. Y. Romanova, et al. “p53 inhibits hypoxia-inducible factor-stimulated transcription,” J. Biol. Chem. 1998; 273: 11995-11998, herein incorporated by reference. Some studies have suggested that inhibition of HIF-1α is linked to the HSP-90 pathway. See e.g., J. S. Isaacs, Y. J. Jung, E. G. Mimnaugh, et al., “Hsp90 regulates a von hippel landau-independent hypoxia-inducible factor-1α-degradative pathway,” J. Biol Chem 2002, 277: 29936-29944, herein incorporated by reference. However, regulation of HIF levels through the ubiquitin/26S proteasome pathway has not previously been proposed.

It remains a priority to determine how HIF-1α is regulated in order to develop effective treatments for HIF control. Regulation of HIF is important in the effective treatment of various conditions related to apoptosis and/or angiogenesis. There is a need to determine the pathway by which HIF-1α is regulated. There is a need to present effective therapeutic treatments for controlling the level of HIF in hypoxic and normoxic cells.

### SUMMARY

One embodiment of the present invention is a therapeutic composition for the regulation of HIF-1α levels in cells under normoxic or hypoxic conditions comprising PX-478. Another embodiment of the present invention is a pharmaceutical formulation comprising PX-478, together with a pharmaceutically acceptable carrier or diluent. In the composition embodiments of the present invention, suitable excipients, carriers, diluents, additives, and/or active ingredients may be used in combination with PX-478. Administration of the therapeutic compositions may be by any suitable means, such as oral, intravenous, systematic, or sustained release.

Another embodiment of the present invention is a method of regulating levels of HIF-1α in cells under normoxic or hypoxic conditions comprising administering to a patient PX-478. Another embodiment of the present invention is a method of decreasing HIF-1α protein levels in cells under normoxic or hypoxic conditions comprising administering to a patient PX-478. Another embodiment of the present invention is a method of decreasing HIF-1 transactivation activity in cells under normoxic or hypoxic conditions comprising administering to a patient PX-478.

Another embodiment of the present invention is a method of regulating HIF-1α degradation by the 26S proteasome in cells under normoxic or hypoxic conditions comprising administering to a patient PX-478. Another embodiment of the present invention is a method of increasing ubiquitination of HIF-1α in cells under normoxic or hypoxic conditions comprising administering to a patient PX-478. Another embodiment of the present invention is a method of inhibiting the deubiquitination of HIF-1α in cells under normoxic or hypoxic conditions comprising administering to a patient PX-478.

Administering of PX-478 does not affect the levels of other cellular proteins, including HSP-90 client proteins, cyclin B1, mutant p53 and histone H1. PX-478 may be administered locally, orally or systemically. PX-478 may be administered in a pharmaceutical formulation together with a pharmaceutically acceptable carrier or diluent, such as water for injection, buffered aqueous solutions, and powdered salts. PX-478 may be administered in a dosage of about 0.001 mg per kg to about 1000 mg per kg body weight of the patient.

### BRIEF DESCRIPTION OF THE FIGURES

Various aspects and applications of the present invention will become apparent to the skilled artisan upon consideration of the brief description of the figures and the detailed description of the invention, which follows:

FIG. 1 illustrates the structure of PX-478.

FIG. 2 illustrates that PX-478 decreases HIF-1α protein levels leading to decreased HIF-1 transactivation activity and expression of downstream target genes.

FIG. 3 illustrates that PX-478 does not affect localization of HIF-1α.

FIG. 4 illustrates that PX-478 increases ubiquitination and degradation of HIF-1α protein.

FIG. 5 illustrates that PX-478 decreases HIF-1α independently of the pVHL and p53 pathways.

FIG. 6 illustrates that PX-478 does not affect levels of HSP-90 client proteins or other proteins controlled by ubiquitination.

FIG. 7 illustrates that PX-478 shows similar effects in vitro and in vivo through Western blotting.

FIG. 8 illustrates that PX-478 affects deubiquitination of HIF-1α.

FIG. 9 is a schematic of the ubiquitin/P26 proteasome mechanism.

### DETAILED DESCRIPTION

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims.

It must also be noted that as used herein and in the appended claims, the singular forms “a”, “an”, and “the” include plural reference unless the context clearly dictates otherwise. Thus, for example, reference to an “enzyme” is a reference to one or more enzymes and equivalents thereof known to those skilled in the art, and so forth. Unless defined otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein are incorporated by reference in their entirety. Nothing herein is to be construed as an admission that the invention is not entitled to antedate such disclosure by virtue of prior invention.

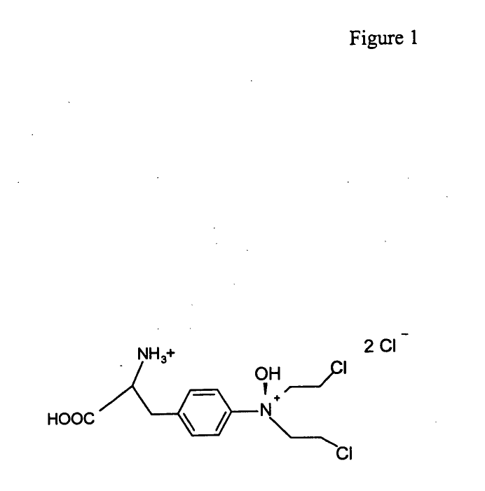

One embodiment of the present invention is a therapeutic composition for the regulation of HIF-1α levels in cells under normoxic or hypoxic conditions comprising PX-478. PX-478 is also known as S-2-amino-3-[4′-N,N,-bis(2-chloroethyl)amino]phenyl propionic acid N-oxide dihydrochloride and its structure is illustrated in FIG. 1.

Embodiments of the invention relate to pharmaceutical formulations containing PX-478. The formulation may also comprise one or more of such compounds together with one or more of a pharmaceutically acceptable carrier, a diluent, an aqueous solution, an adjuvant, or another compound useful in treating a patient in need thereof. Suitable formulations may include buffered solutions, water for injection, or powdered salts. A pharmaceutical formulation comprising one or more of the compounds may be administered as intravenous infusion. The invention includes a method of medical treatment comprising the use of such compounds. The method may also comprise using such compounds together with other methods of medical treatment useful in treating particular diseases, such as radiotherapy or chemotherapy.

Other embodiments include pharmaceutical formulations containing therapeutic compositions of the present invention. The formulation may comprise one or more of therapeutically effective compounds together with one or more of a pharmaceutically acceptable carrier, a diluent, an aqueous solution, an adjuvant, an excipient, an additive, or another compound useful in treating various medical conditions.

As used herein, the term “therapeutic” refers to the ability of a compound to effect protein levels within cells under hypoxia (about 1% oxygen) or normoxia (about 20% oxygen). Specifically, the ability to decrease levels of HIF-1, and more specifically HIF-1α in cells under hypoxia or normoxia is found in the several compounds of the present invention.

The invention also relates to salts of the above compounds illustrated in FIG. 1. The salt would generally have the formulas as set out in FIG. 1 with the addition of a salt, wherein the salt may be preferably any of HCI, acetate, TFA, tosylate or picrate.

Excipients or stabilizers may be used in connection with the therapeutic compounds of the present invention. Stabilizers include carbohydrates, amino acids, fatty acids, and surfactants and are known to those skilled in the art.

A therapeutic compound of the present invention may be administered orally, locally or systemically. Such administration includes oral, parenteral, enteral, intraperitoneal, intrathecal, inhalation, or topical administration. The preferred forms of administration include oral, intravenous, subcutaneous, intrathecally, intradermal, intramuscular, internodal, intracutaneous, or percutaneous. Topical or local administration may preferable for greater control of application. Suitable dosage levels may be administered. A dosage of about 0.001 mg per kg to about 1000 mg per kg body weight of the patient may be administered. In previous studies, PX-478 has been subjected to various toxicity studies and suitable dosages have been determined.

PX-478, singularly or in combination with other active ingredients, can be mixed with an appropriate pharmaceutical carrier prior to administration. PX-478 may be administered in a pharmaceutical formulation together with a pharmaceutically acceptable carrier or diluent, such as water for injection, buffered aqueous solutions, and powdered salts. Examples of generally used pharmaceutical carriers and additives are conventional diluents, binders, lubricants, coloring agents, disintegrating agents, buffer agents, isotonizing fatty acids, isotonizing agents, preservants, anesthetics, surfactants and the like, and are known to those skilled in the art. Specifically pharmaceutical carriers that may be used are dextran, sucrose, lactose, maltose, xylose, trehalose, mannitol, xylitol, sorbitol, inositol, serum albumin, gelatin, creatinine, polyethlene glycol, non-ionic surfactants (e.g. polyoxyethylene sorbitan fatty acid esters, polyoxyethylene hardened castor oil, sucrose fatty acid esters, polyoxyethylene polyoxypropylene glycol) and similar compounds. Pharmaceutical carriers may also be used in combination, such as polyethylene glycol and/or sucrose, or polyoxyethylene sorbitan fatty acid esters, polyoxyethylene sorbitan monooleate. The release profiles of such formulations may be rapid release, immediate release, controlled release or sustained release.

Another aspect of the present invention, is the treatment of diseases by inhibiting HIF, particularly HIF-1α. Just a few of the diseases that may be treated with the compounds, compositions and formulation of the present invention include diseases associated with angiogenesis, neovascularization or apoptosis. Diseases associated with HIF which may be treated include choroidal and retinal neovascularization, age-related macular degeneration, joint disease, inflammation, nuerodegenerative diseases, and ischemic neperfusion injury, and other disease as described herein.

Another embodiment of the present invention is a method of regulating levels of HIF-1α in cells under normoxic or hypoxic conditions comprising administering to a patient PX-478. Another embodiment of the present invention is a method of decreasing HIF-1α protein levels in cells under normoxic or hypoxic conditions comprising administering to a patient PX-478. Another embodiment of the present invention is a method of regulating HIF-1α degradation by the 26S proteasome in cells under normoxic or hypoxic conditions comprising administering to a patient PX-478. Another embodiment of the present invention is a method of decreasing HIF-1 transactivation activity in cells under normoxic or hypoxic conditions comprising administering to a patient PX-478.

Another embodiment of the present invention is a method of increasing ubiquitination of HIF-1α in cells under normoxic or hypoxic conditions comprising administering to a patient PX-478. Another embodiment of the present invention is a method of increasing polyubiquitinated HIF-1α levels in cells under normoxic or hypoxic conditions comprising administering to a patient PX-478. Another embodiment of the present invention is a method of inhibiting the deubiquitination of HIF-1α in cells under normoxic or hypoxic conditions comprising administering to a patient PX-478.

In the method embodiments of the present invention, the administering of PX-478 does not affect the levels of other cellular proteins, including HSP-90 client proteins, cyclin B1, mutant p53 and histone H1.

Mammalian cells have developed an elaborate array of adaptive responses to maintain oxygen homeostasis. The most important mediator of the cellular response to hypoxia identified to date is the hypoxia-inducible factor-1 (HIF-1) transcription factor. HIF-1 transcriptionally activates a number of genes encoding proteins that play crucial roles in the acute and chronic adaptation to oxygen deficiency. These genes encode proteins involved in erythropoiesis, glycolysis, promotion of cell survival, resistance to apoptosis, inhibition of cell differentiation, and promotion of angiogenesis. HIF-1 has been implicated in a wide range of human diseases including cancer, ischemic myocardial and limb disease, ischemic stroke, Alzheimer's disease, neurodegeneration and age-related macular degeneration. Modulation of HIF-1 signaling has been proposed as a potential therapeutic strategy for these diseases.

HIF-1 is a heterodimer consisting of an oxygen regulated alpha subunit (HIF-1α, HIF-2α or HIF-3α) and a constitutively expressed beta subunit (HIF-1β, also known as the Aryl Hydrocarbon Nuclear Translocator, or ARNT). Both subunits are members of the basic-helix-loop-helix (bHLH)-PER-ARNT-SIM (PAS) superfamily of eukaryotic transcription factors, which bind to DNA via basic domains and form dimeric complexes via HLH domains. The regulation of HIF-1 transcriptional activity by oxygen is mediated through the HIF-1α subunit. HIF-1α mRNA is constitutively translated but under normoxic conditions the activity of HIF-1 is kept very low due to the rapid breakdown of HIF-1α by the ubiquitin/26S proteasome pathway. Under normoxic conditions a family of HIF-1α-directed proline (Pro) hydroxylases convert Pro⁴⁰² and Pro⁵⁶⁴ in the oxygen degradation domain (ODD) of human HIF-1α to hydroxyproline. These modifications allow HIF-1α to be recognized by the E3 ubiquitin ligase complex containing the von Hippel-Lindau tumor suppressor protein (pVHL). This complex catalyzes the polyubiquitination of HIF-1α, thus, targeting it for rapid degradation by the 26S proteasome. The HIF-1 prolyl hydroxylases are dioxygenases that show an absolute requirement for oxygen, Fe²⁺ and 2-oxoglutarate as cofactors. Under oxygen deprived conditions, or when HIF-1 prolyl hydroxylases are inactivated by agents such as Co²⁺, iron chelators, or 2-oxoglutarate-mimetic compounds, the HIF-1α prolines remain unmodified leading to stabilization of HIF-1α and the formation of HIF-1α/HIF-1β heterodimers (HIF-1). The HIF-1 complex then undergoes additional post-translational modification and is translocated to the nucleus, where it binds to hypoxic response element (HRE) in the promoter regions of HIF-1-responsive genes to activate their transcription.

Thus, cellular HIF-1α levels increase under hypoxic conditions. Many human cancers also show constitutively increased HIF-1α levels, even at normoxic conditions such as PC-3 prostate cancer cells and RCC4 renal cancer cells.

Several recent studies have shown that levels of HIF-1α protein may also be regulated independently of the pVHL pathway although the precise mechanism has not been determined in the art. Some studies have suggested that HIF-1α is degraded by the 26S proteasome pathway following ubiquitination.

Applicants have found that a novel small molecule anticancer agent, PX-478 (FIG. 1), is a suppressor of HIF-1α protein levels and an inhibitor of HIF-1 signaling. The Applicants have found that this occurs with an increased polyubiquitination of HIF-1α and increased HIF-1α degradation, independent of other known pathways of HIF-1α regulation. Applicants have found that inhibition of HIF-1α by PX-478 is associated with inhibition of a cytoplasmic HIF-1 deubiquitinase activity.

PX-478 (S-2-amino-3-[4′-N,N,-bis(2-chloroethyl)amino]phenyl propionic acid N-oxide dihydrochloride) or melphalan N-oxide and derivatives thereof significantly decrease the hypoxia-induced increase in HIF-1α protein, inhibits HIF-1 transactivation and decreases the expression of the downstream target genes such as vascular endothelial growth factor (VEGF) and inducible nitric oxide synthase (iNOS), in several cancerous cell lines.

In the present method embodiments, human renal cell carcinoma (RCC4 cells) lacking active von Hippel Lindeau protein (pVHL) and RCC4/VHL cells into which active pVHL has been reintroduced were used to show that PX-478 acts independently of the pVHL pathway. Von Hippel Lindeau protein (pVHL) has been thought to regulate the breakdown of HIF-1α.

HIF-1α protein is found in a wide variety of human primary tumors but only at very low levels in normal tissue. The importance of HIF-1α to cancer is demonstrated by the high incidence of tumors such as renal cell carcinoma, pheochromocytoma and hemingioblastoma of the central nervous system in individuals with loss of function of both alleles of the VHL gene leading to elevated HIF-1α levels. In addition, most cases of sporadic renal cell carcinoma are associated with an early loss of function of the VHL gene and increased HIF-1α levels. Reintroduction of the intact VHL gene into cells derived from renal carcinomas restores HIF-1α to normoxic levels and decreases tumorigenicity. HIF-1α levels are also increased in cancer cells with mutant or deleted PTEN. HIF-2α which is expressed in some tumors is also found in bone marrow and tumor associated macrophages.

Because of the role of HIF-1α in regulating the response of growing tumors to hypoxia it is a very important target for anticancer drug development. U.S. Pat. No. 5,602,278, herein incorporated by reference in its entirety, describes PX-478 as an agent that would be selectively activated in hypoxic environments. PX-478 was shown to preferentially kill hypoxic cells in a reducing environment (e.g. in the presence of a reducing enzyme).

In method embodiments, Applicants have investigated the effects of PX-478 on HIF-1α and its downstream targets due to its antitumor effect in the absence of reducing enzymes. In method embodiments, PX-478 treatment leads to a decrease in HIF-1α protein (both in vitro and in vivo) and subsequent transactivation of the HIF-1 complex leading to decreased levels of downstream targets, possibly through inhibition of thioredoxin-reductase. In method embodiments, the activity of PX-478 is independent of the VHL pathway. In method embodiments, PX-478 inhibits HIF-1α without requiring reducing enzymes.

In method embodiments, PX-478 suppresses HIF-1α protein levels in cancer cells with constitutive expression of HIF-1α under aerobic conditions (PC-3 prostate cancer and RCC4 renal cancer) and in other cancer cells that only show increased HIF-1α under hypoxic conditions (MCF-7 breast cancer, HT-29 colon cancer and HCT-116 colon cancer). HIF-1α has been shown to localize to the nucleus during activation of the HIF-1 pathway. In method embodiments, there is no affect caused by PX-478 in the localization or the inhibition of HIF-1α in nuclear and cytoplasmic fractions. In method embodiments, a decrease in HIF-1α caused by PX-478 under hypoxic conditions may be associated with increased degradation of HIF-1α measured by immunoblotting in the presence of cycloheximide to block new protein synthesis, and by a decreased half life of HIF-1α. Through the administration of PX-478, the half life of HIF-1α, may decrease from about 3 hours to about 1.5 hours as measured by pulse chase experiments with [³⁵S]-labeled methionine/cysteine.

PX-478 regulates HIF-1α levels independently of the well-characterized pVHL pathway. pVHL binds to the oxygen degradation domain (ODD) of HIF-1α which recruits a ubiquitin-protein ligase complex containing elongin B, elongin C and cullin resulting in ubiquitination of HIF-1α and degradation by the 26S proteasome. Binding of pVHL is mediated by hydroxylation of Pro⁴⁰² and Pro⁵⁶⁴ in the ODD of human HIF-1α by prolyl-4-hydroxylases (PHDs). PHDs are dioxygenases that show an absolute requirement for oxygen, Fe ²⁺ and 2-oxoglutarate as cofactors. Under oxygen-deprived conditions, or when PHDs are inactivated by competitive substrate analogues, the HIF-1α prolines remain unmodified preventing binding of pVHL and, consequently, HIF-1α levels increase. The levels of hydroxyPro⁵⁶⁴ HIF-1α relative to HIF-1α were not decreased by PX-478 under hypoxic conditions. HIF-1α levels were decreased by PX-478 in RCC4 lacking functional VHL and in RCC4/VHL cells to which VHL was reintroduced-showing that the effect of PX-478 is not mediated by the hydroxy proline/pVHL pathway.

Another pathway controlling HIF-1α levels is mediated by the tumor suppressor p53 and MDM2. The MDM2 ubiquitin protein ligase is recruited to HIF-1α by binding of p53 which results in a decrease in HIF-1α levels by MDM-2 mediated ubiquitination and proteasomal degradation of HIF-1α. This may explain why the loss of p53 in tumor cells enhances HIF-1α levels. See for example, R. Ravi, B. Mookerjee, et al., “Regulation of tumor angiogenesis by p53-induced degradation of hypoxia-inducible factor 1 alpha,” Genes Develop 2000; 14(1):34-44, herein incorporated by reference. In method embodiments, PX-478 does not promote HIF-1α degradation through a p53 dependent mechanism. A similar inhibition of HIF-1α levels by PX-478 may be observed in MCF-7 cells, which have wild type p53, and HT-29 and PC-3 cells which have mutant p53. Similar effects of PX-478 on HIF-1α levels may be observed in HCT116+/+ human colon carcinoma cells expressing wild type p53 and HCT116−/− cells from which p53 has been deleted by homologous recombination. Thus, the effects of PX-478 do not involve the p53/MDM2 pathway of degradation.

Recent studies have suggested another pVHL independent pathway for degradation of HIF-1α involving the heat-shock protein-90 (HSP-90). The HSP-90 inhibitors geldanamycin (GA) and 17-allylamino-17-desmethoxygeldanamycin (17-AAG) promote the loss of HIF-1α protein from several cell-lines lacking pVHL. Mutation of proline residues Pro⁴⁰² and Pro⁵⁶⁴ in HIF-1α failed to protect HIF-1α from GA-induced degradation. We found that PX-478 did not affect levels of the HSP-90 or its client proteins: Akt, Src, and Raf-1 demonstrating that the HSP-90 pathway is not involved in the effects of PX-478.

The decrease in HIF-1α caused by PX-478 is associated with an increase in levels of ubiquitinated HIF-1α. Ubiquitination is a necessary step for proteasomal degradation by marking proteins for degradation by the 26 proteasome. Refer to FIG. 9, which illustrates the ubiquitin/P26 proteasome mechanism. Ubiquitination is an important post-translational modification that controls the steady-state levels of numerous key regulatory molecules in the cell and/or influences their localization and function. See, for example, R. Hartman-Petersen, M. Seeger, C. Gordon, “Transferring substrates to the 26S proteasome,” Trends. Biochem. Sci. 2003; 28: 26-31, herein incorporated by reference. The ubiquitinating enzymes that link ubiquitin to proteins are relatively well characterized. Conjugation is initiated by the activation of ubiquitin by the ubiquitin-activating enzyme, E1, which forms a high-energy ubiquitin-thiol ester bond in the presence of ATP. It then transfers the activated ubiquitin to an ubiquitin-conjugating enzyme, E2, forming an E2-thiol ester bond. Finally, ubiquitin is transferred to a target substrate protein through an isopeptide linkage between the conserved C-terminal glycine residue of ubiquitin and the c-amino group of the lysine residue of the substrate, involving an ubiquitin ligase, E3. Sequential conjugation of the internal lysine residue of ubiquitin to a C-terminal glycine residue of a new ubiquitin molecule results in formation of a polyubiquitin chain, which targets proteins for degradation by the 26S proteasome. Refer to FIG. 9.

The increase in levels of ubiquitinated HIF-1α by PX-478 presumably reflects a steady state increase despite an increased rate of HIF-1α degradation. The increased ubiquitination caused by PX-478 is specific for HIF-1α since an increase in total protein ubiquitination is not seen after administration of PX-478. A change in the levels of other proteins whose levels are regulated by ubiquitination including HSP-90 client proteins, and cyclin B1, mutant p53 and histone H1 is not observed after administration of PX-478.

In method embodiments, the suppressive effect of PX-478 on HIF-1α accumulation demonstrates that PX-478 interferes with the deubiquitination of HIF-1α in vivo and in vitro. More than 90 deubiquiting enzymes have been identified by the human genome project and this large number strongly suggests that, in vivo, these enzymes have specific substrates and regulatory activities. The specificity is likely to largely depend on the structure of target proteins but may also depend on accessibility of the ubiquitinated proteins to deubiquitinating enzymes, as well as the length and types of polyubiquitin chains and the abundance and localization of both deubiquitinases and target proteins. Deubiquitinating enzymes can be divided into two well-defined classes on the basis of sequence homology—ubiquitin carboxy-terminal hydrolases (UCH) and ubiquitin processing proteases (UBPs). Both classes of enzymes contain highly conserved sequence motifs containing cysteine, histidine and aspartic acid residues, exhibit nearly identical geometry at their active sites and employ a highly conserved catalytic mechanism for deubiquitination of target proteins. Substrate specificity is may result from the highly variable sequences found outside of the active site, particularly in UBPs. Mutation of any of the catalytic triad residues (Cys, His, and Asp) or residues that compromise the oxyanion hole (Asn, Asn, and Asp) results in loss of enzymatic activity.

Inactivation of the active site cysteine residue by alkylation with iodoacetamide also completely abolishes enzyme activity. Iodoacetamide prevents deubiquitination of HIF-1α. In method embodiments, PX-478 inhibits HIF-1α by deubiquitination. There may be direct inhibition of a deubiquitinase by PX-478, or indirect inhibition of a deubiquitinase enzyme by interferences of a protein up-stream responsible for maintaining the activity of the deubiquitinase. The use of PX-478 may be targeted against the proteins that mediate the removal of ubiquitin from HIF-1α and that favor the accumulation of HIF-1α under both normoxic and hypoxic conditions. Overexpression of the HIF-1α-targeted deubiquitination machinery in cancer cells may contribute to the deregulated activation of this pathway during tumor progression.

PX-478 is an anticancer agent and has shown antitumor activity against a number of human tumor xenografts. In method embodiments, treatment of mice bearing HT-29 human tumor xenografts with PX-478 results in a suppression of tumor HIF-1α, and an increase in the levels of ubiquitinated HIF-1α without changes in the levels of other proteins known to be degraded by the ubiquitin/proteasome pathway.

PX-478 suppresses both constitutive and hypoxia-induced HIF-1α protein levels and HIF-1 transactivation in cancer cells in vitro and in vivo . The effect of PX-478 is independent of the pathways of HIF-1α regulation involving pVHL, p53 or HSP-90 and is associated with an increase in polyubiquitinated HIF-1α. This is due to inhibition of HIF-1α deubiquitination by PX-478.

The results have implications for the design of novel therapeutic strategies based on modulation of the deubiquitination system for a range of other proteins. The deubiquitination system may be a general mechanism for control of protein levels. The demonstration that HIF-1α can be selectively inhibited by affecting its deubiquitination suggests that deubiquitinases exhibit sufficient substrate specificity that small molecules can be developed to selectively alter ubiquitination and degradation of any target molecule regulated by this pathway. This mechanism therefore has the potential to influence therapy for a wide range of human diseases.

Practice of the invention, including additional preferred aspects and embodiments thereof, will be more fully understood from the following examples and discussion, which are presented for illustration only and should not be construed as limiting the invention in any way.

### Materials, Examples and Discussion

Cells treatments—MCF-7 human breast cancer, HT-29 colon cancer and PC-3 human prostate cancer cells were obtained from the American Tissue Type Collection (Rockville, Md.). Human renal cell carcinoma RCC4 cells and RCC4/VHL into which the wild-type von Hippel-Lindau (pVHL) gene has been transfected were obtained from Dr. P. Ratcliffe (Welcome Trust Centre for Human Genetics, Oxford, UK). Human colon carcinoma HCT116+/+ cells and HCT116−/− from which the p53 gene has been deleted by homologous recombination were obtained from Dr B. Vogelstein (Johns Hopkins, Baltimore, Md.).

Cells were grown under humidified 95% air, 5% CO₂ at 37° C. in Dulbecco's modified Eagle's medium (HT-29, MCF-7, RCC4, RCC4/VHL, HCT116+/+ and HCT116−/− cells) or Ham's F12 media (PC-3 cells) supplemented with 10% fetal bovine serum (FBS), and 1 mg/ml G418 for the RCC4 and RCC4/VHL cells. Cells were treated, as indicated, with the following agents: 10 M N-carbobenzoxy-Leu-Leu-norvalinal (LLnV, MG115; Sigma Chemical Co., St. Louis, Mo.), 40 g/ml cycloheximide (Sigma) and PX-478 (S-2-amino-3-[4′-N,N,-bis(2-chloroethyl)amino]phenyl propionic acid N-oxide dihydrochloride; FIG. 1) (ProlX Pharmaceuticals, Tucson, Ariz.) at various concentrations.

Hypoxic treatment—Cells in culture flasks were placed for various times in a humidified chamber at 37° C., maintained at 1% oxygen in the gas phase using a calibrated oxygen sensor (Pro:Ox 110, Biospherix, Redfield, N.Y.) that adds oxygen to a pre-mixed mixture of 5% CO₂/74% N₂/21% argon. Buffers and medium, where indicated, were pre-equilibrated by incubation for 16 hours in the chamber.

VEGF ELISA—Human VEGF in cell lysates and VEGF secreted into the growth medium was measured using an ELISA kit that detects VEGF₁₆₅ and VEGF₁₂₁ isoforms (Human VEGF-ELISA; R&D Systems, Minneapolis, Minn.) as described previously. VEGF in cell lysates was expressed as pg VEGF protein/mg of total cell protein and VEGF in the medium corrected to pg VEGF protein/mg of total cell protein measured in cells from the same flask.

Hypoxia Response Element Reporter Assay—The pGL3 firefly luciferase reporter plasmid containing the hypoxia response element (HRE) from phosphoglycerate kinase (PGK) (22) was supplied by Dr I. Stratford (University of Manchester, UK). Plasmid DNA was prepared using a commercial kit (Qiagen, Valencia, Calif.). The empty pGL3 control plasmid and the pRL-CMV Renilla luciferase containing plasmid used to control for transfection efficiency were obtained from Promega (Madison, Wis.). Cells were transfected with 5 μg of HIF-1α reporter plasmid or pGL3 control plasmid, and 0.025 μg pRL-CMV Renilla luciferase plasmid using LipoTAXI mammalian transfection reagent (Stratagene, La Jolla, Calif. Twenty four hours later cells were exposed to hypoxia for 16 hr as previously described. Firefly and Renilla luciferase activity was measured using the Dual-Luciferase Reporter Assay System (Promega, Madison, Wis.) according to the manufacturer's instructions.

Immunoblotting analysis—Nuclear and cytoplasmic extracts were prepared using NE-PER™ Nuclear and Cytoplasmic Extraction Reagents (Pierce, Rockford, Ill.) according to the manufacturer's instructions. Protein concentration was determined using the Biorad Protein Assay (Biorad) according to the manufacturer's instructions. Western blotting was performed as described previously using mouse anti-human HIF-1α monoclonal antibody (Transduction Labs, Lexington, Ky.) 1 g/ml, mouse anti-human HIF-1α monoclonal antibody (SantaCruz Biotechnology, Santa Cruz, Calif.) 1 g/ml, goat anti-human actin polyclonal antibody (Santa Cruz Biotechnology) 0.5 g/ml, goat anti-human lamin A polyclonal antibody (SantaCruz Biotechnology) 0.5 g/ml, rabbit anti-human AKT (Cell Signaling Technology) 1:1000, rabbit anti-human Raf-1 (Santa Cruz Biotechnology) 0.5 g/ml, mouse anti-human HSP-90 (Stressgen) 0.5 g/ml, rabbit anti-human ubiquitin (Sigma) 1:1000, rabbit anti-human VEGF (Santa Cruz Biotechnology), mouse anti-human p53 (Santa Cruz Biotechnology) 0.5 g/ml, mouse anti-human Src-1 (Upstate Biotechnology) 1 g/ml, mouse anti-human histone H1 (Upstate Biotechnology) 1 g/ml, mouse anti-human cyclin B1 (Upstate Biotechnology) 1 g/ml. The antibody specific for the hydroxylated Pro⁵⁶⁴ residue of HIF-1α was generated by immunizing rabbits with a hydroxy-Pro⁵⁶⁴ containing peptide from HIF-1α (amino acid residues 556-570) coupled to keyhole limpet hemocyanin. The antibody was immunaaffinity purified by binding to the same peptide used for the immunization and used at a concentration of 1 g/ml. In preliminary experiments the hydroxy-Pro⁵⁶⁴ antibody was shown to be specific for the Pro⁵⁶⁴ modified form of HIF-1α and did not recognize the related hydroxy-Pro⁴⁰² site in HIF-1α. Anti-mouse or anti-goat horseradish peroxidase-conjugated secondary antibodies (Amersham Bioscience Corp., Piscataway, N.J.) were used at a dilution of 1:5000 for detection by chemiluminescence and blots were quantified using ImageQuant software (Molecular Dynamics, Sunnyvale, Calif.).

Immunoprecipitations—Following cell lysis and clarification by centrifugation, lmg protein aliquots of lysate were incubated for 30 min at 4° C. with 50 l of a 25% slurry of swelled protein-A Sepharose beads (Sigma Chemical Co). Beads were removed by centrifugation (1000×g, 5 min, 4° C.) and lysates was incubated for 2 h at 4° C. with 10 l mouse anti-human HIF-1α antibody (Transduction Labs). 50 l of a 25% slurry of swelled protein A Sepharose beads was then added overnight at 4° C. before the beads were washed four times with lysis buffer, boiled in sample buffer for 10 min and loaded onto 10% Bis/Tris NuPage gels (Invitrogen). Western blotting was then performed as indicated.

Immunofluorescence—Cells were grown to 70% confluence on 13 mm diameter glass coverslips and exposed to hypoxia for 16 h in the presence of PX-478. Cells were immediately placed on ice and fixed in 4% formaldehyde in phosphate buffered saline (PBS) for 15 min. Cells were permeabilized in 0.1% Triton-X-100 in PBS for 10 min and then blocked for 1 h in 10% fetal bovine serum in PBS. RNA was then removed by incubation at 37° C. for 1 h in 10 mg/ml RNase in PBS. Cells were incubated overnight at 4° C. with mouse anti-human HIF-1α antibody (Transduction Labs) 0.5 μg/ml in PBS and HIF-1α was visualized using an anti-mouse secondary antibody conjugated to Alexa dye 488 (Molecular Probes, Eugene,Oreg.), diluted 1:1000 in PBS. DNA was stained using 200 nM BOBO3 dye (Molecular Probes), diluted in PBS. Coverslips were then mounted on slides using Vectashield Hard Mount (Vector Laboratories, Burlingame, Calif.) and examined using a Nikon TE300 inverted microscope equipped for epiflourescence (A. G. Heinze, Chandler, Ariz.) and imaged with a Roper Coolsnap digital camera (Princeton Instruments, Trenton, N.J.).

Pulse-Chase Analysis—Pulse-chase analysis was carried out as described by Isaacs et al (17). Briefly, exponentially growing cells were starved for 30 min in methionine and cysteine-free media (Invitrogen, Carlsbad, Calif.) and 150 Ci/ml methionine/cysteine (Tran³⁵S-label, ICN Radiopharmaceuticals, Irvine, Calif.) was added for 1 hour. After the labeling period, cells were washed with non-radioactive complete medium (chase medium) and incubated for the indicated times. Cells were then lysed and pre-cleared with protein A Sepharose beads and HIF-1α was immunoprecipitated from lmg of soluble lysate protein overnight as described above. For PX-478 treated samples, PX-478 was added to the chase media. Blots were quantified using ImageQuant software (Molecular Dynamics).

Deubiquitination assay—Deubiquitination of HIF-1α was measured using a modified assay of the assay described in Strayhorn and Wadsinski (Strayhorm W. B., Wadziniski, B. E., A Novel in Vitro Assay for Deubiquitination of I kappa B alpha. Arch. Biochem. Biophys. 2002; 400:76-84, herein incorporated by reference). To prepare poly-ubiquitinated HIF-1α as substrate for the assay, HT-29 cells were exposed to partial hypoxia for 16 hours in the presence of 5 μM LLnV to inhibit proteasomal degradation, allowing accumulation of polyubiquitinated forms of HIF-1α. Cells were thoroughly washed in ice-cold PBS and lysed in lysis buffer A (20 mM Tris, pH 7.5, 150 mM NaCl, 0.1% Triton-X-100, and 0.2% NP-40) supplemented with fresh protease inhibitors (1 μg/ml pepstatin, 3 μg/ml aprotinin, 20 μM leupeptin, 1 mM PMSF, 1 mM EGTA, and 1 mM EDTA), phosphatase inhibitors (10 mM sodium pyrophosphate, 10 mM NaF, and 0.4 mM sodium orthovanadate), and 5 mM iodoacetamide to inhibit endogenous deubiquitinating enzymes. Cell lysates were centrifuged at 15,000×g at 4° C. for 5 min and the pellet was discarded. The protein concentration of clarified lysates was determined and lysates diluted to 2 mg protein/ml in lysis buffer A. Immunoprecipitation of HIF-1α was then performed as described above. The immune complexes were washed four times in lysis buffer A and four times in reaction buffer A (20 mM Tris, pH 7.5, 150 mM NaCl, 0.1% Triton-X-100, and 0.2% NP-40 supplemented with 5 mM dithiothreitol, 1% bovine serum albumin, phosphatase inhibitors, and protease inhibitors without leupeptin). It was typically necessary to readjust the pH of lysis buffer A and reaction buffer A to 7.5 after all of the components were added.

To prepare cell extracts as a source of deubiquitinating enzyme activity for the assay, HT-29, PC-3 or MCF-7 cells were grown to 70% confluency and washed in ice-cold PBS. Cells were lysed in ice-cold lysis buffer B (20 mM Tris, pH 7.5, 150 mM NaCl, 0.1% Triton-X-100, and 0.2% NP-40 supplemented with 1 mM dithiothreitol, phosphatase inhibitors, and protease inhibitors without leupeptin) for 30 min and then centrifuged at 15,000×g at 4° C. for 5 min. The pellet was discarded and protein concentration was determined. Aliquots of lysate were snap frozen in liquid nitrogen and stored at −80° C. Approximately 15 min prior to assay, lysates were diluted to 3 mg protein/ml in lysis buffer B, and dithiothreitol was added to a final concentration of 5 mM. For heat inactivation, lysates were heated to 95° C. for 5 min and placed back on ice. For treatment with drugs, lysates were incubated for 15 min on ice with 15 mM iodoacetamide, 10 μM LLnV, or PX-478 at the concentrations indicated. For reactions containing iodoacetamide, the pre-incubation was allowed to proceed with only 1 mM dithiothreitol to avoid reduction of the chemical inhibitor.

To measure deubiquitination of HIF-1α, 50 μl of enzyme lysate was added to 50 μl of a 25% slurry of HIF-1α substrate bound to protein A Sepharose beads and incubated at 37° C. for 1 hour. The mixture was then placed on ice and the beads were washed twice with lysis buffer A before boiling in 40 μl sample buffer and the reaction products detected by Western blotting using anti-ubiquitin antibody.

Animal studies—10⁷ HT-29 colon tumor cells were injected subcutaneously into the flanks of male severe combined immunodeficient (scid) mice and tumors allowed to grow until they were approximately 300 mm³. The mice were then treated intraperitoneally with saline vehicle or 100 mg/kg PX-478 and 2 hr later the animals killed, the tumors removed, homogenized in lysis buffer A (see above), snap frozen in liquid nitrogen and stored frozen at −80° C.

### EXAMPLE 1

### PX-478 Inhibits HIF-1αProtein and HIF-1 Signaling

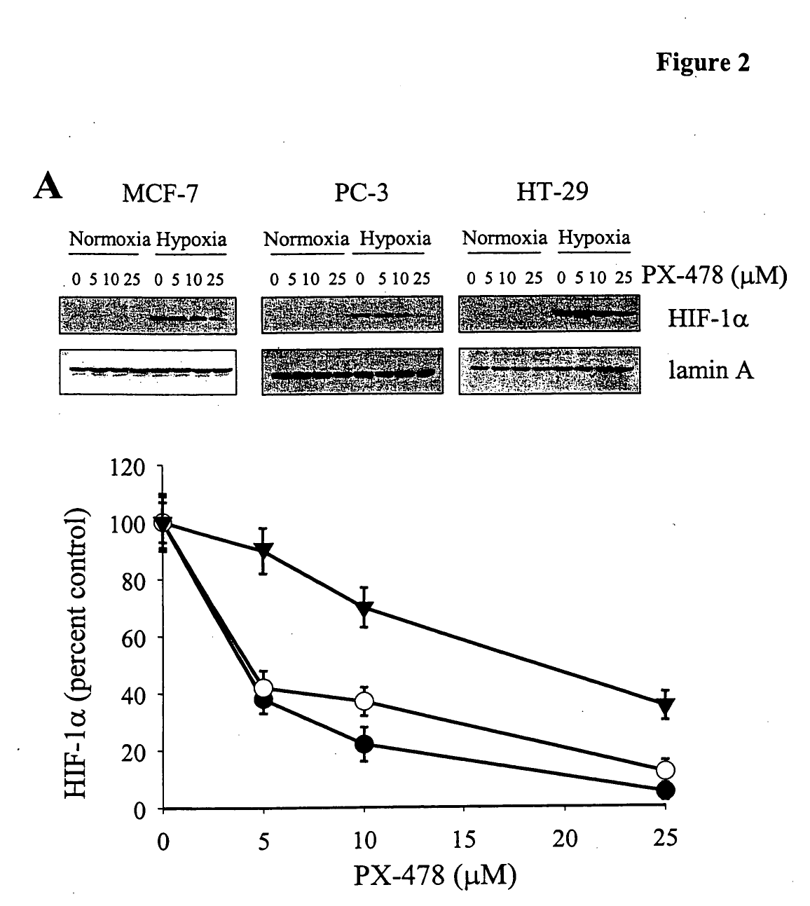

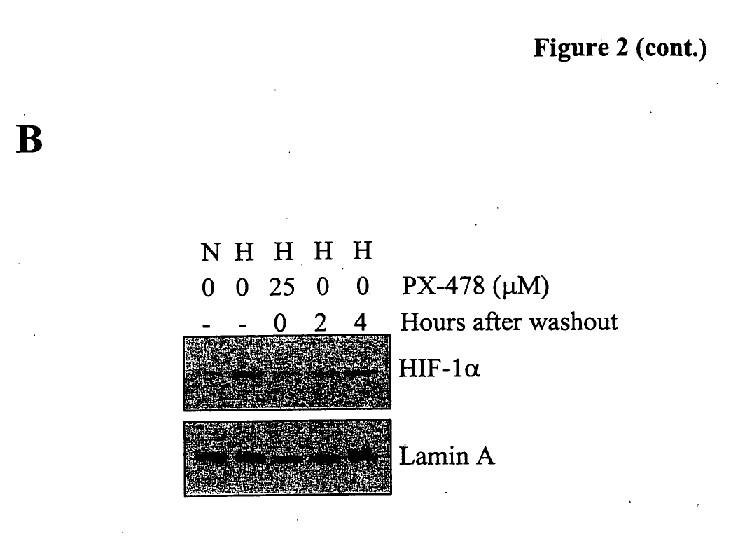

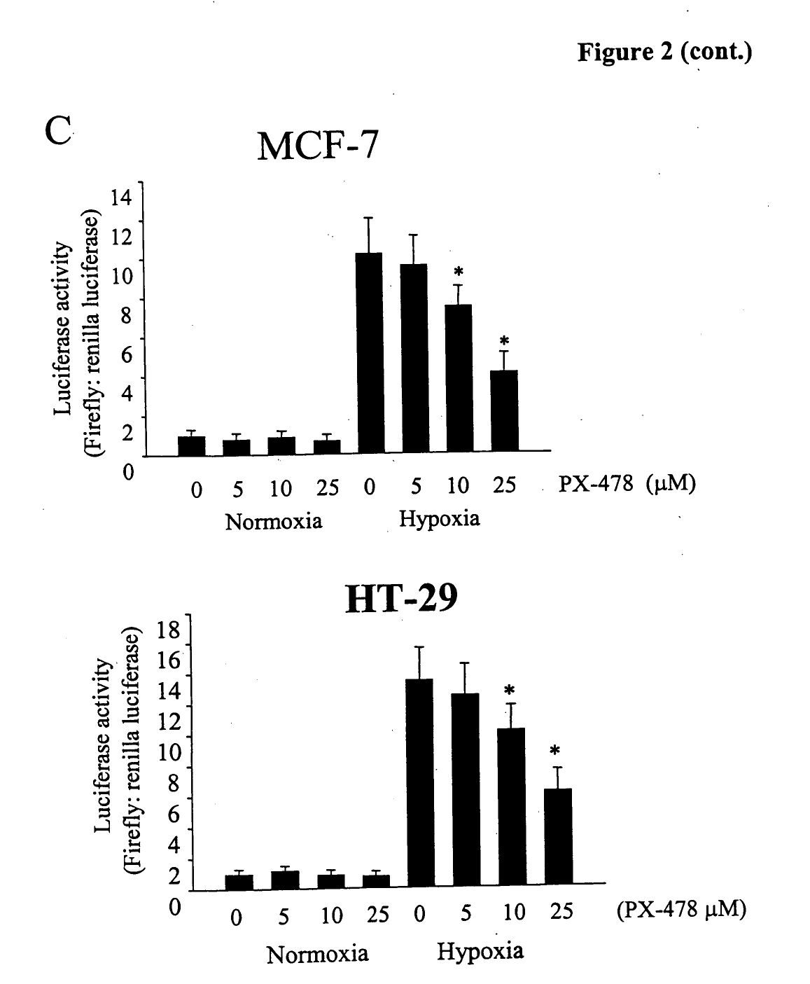

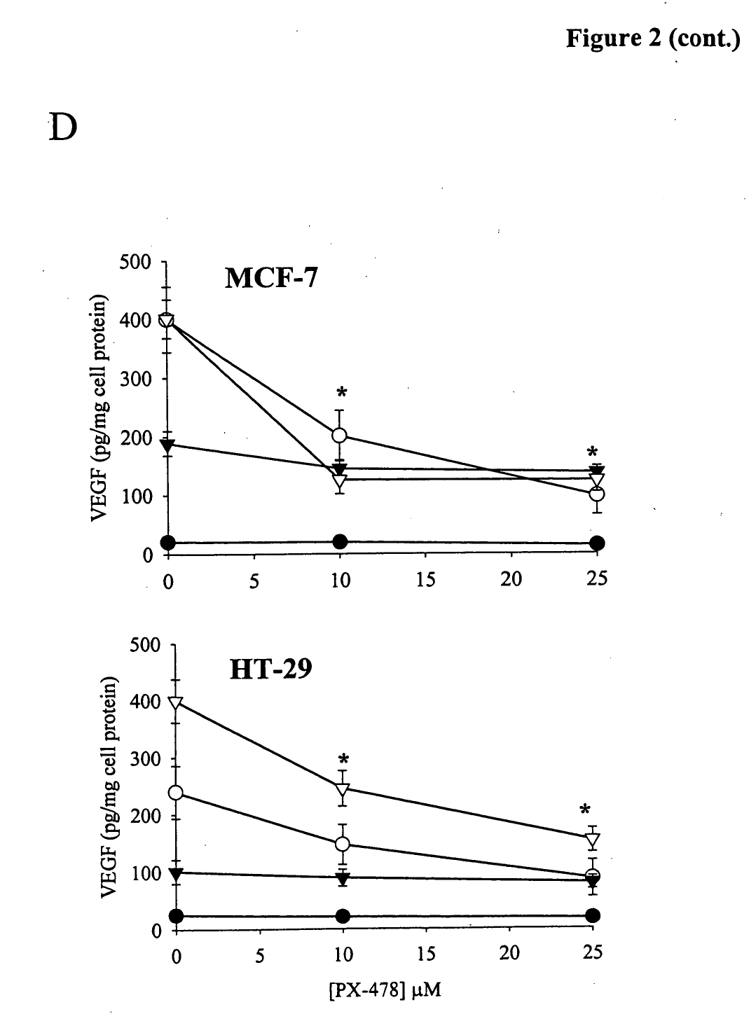

In FIG. 2, human breast cancer MCF-7, colon cancer HT-29, and prostate cancer PC-3, under hypoxic conditions were treated with PX-478. A. Human breast cancer MCF-7, colon cancer HT-29 and prostate cancer PC-3 cells were treated for 16 hours with various doses of PX-478, as indicated, under normoxic (20% oxygen) or hypoxic (1% oxygen) conditions. Nuclear extracts were prepared and HIF-1α protein levels were examined using Western blotting (top panel). Blots were quantitated using Image Quant (lower panel). Lamin A was used as a loading control. B. MCF-7 cells were exposed to hypoxia (H) for 16 hours in the presence of 25 M PX-478, drug was washed out and recovery of HIF-1α levels under conditions of hypoxia was examined after the times indicated using Western blotting on nuclear extracts. Cells were also treated for 16 hours under normoxia (N) or hypoxia (H), in the absence of PX-478, as controls. Lamin A was used as a loading control. C. MCF-7 and HT-29 cells were transiently transfected with vectors expressing either Firefly luciferase under the control of multiple copies of the HRE from PGK or constitutively expressing renilla luciferase to control for transfection efficiency. Cells were exposed to normoxia or hypoxia for 16 hours in the presence of PX-478 as indicated. Luciferase activity was expressed as the ratio of Firefly: renilla luciferase. D. MCF-7 and HT-29 cells were treated for 16 hours as indicated with PX-478 under normoxia (closed symbols) or hypoxia (open symbols). VEGF protein was measured in total cell lysates (circles) and that secreted into the medium (triangles) using an ELISA kit. VEGF levels were expressed per mg.

Cells exposed to hypoxia (1% oxygen) for 16 hours showed a large increase in HIF-1α protein measured by Western blotting without significant cell death. Exposure to lower concentrations of 0.1% oxygen for 16 hours produced more than 30% cell death; therefore, all hypoxia studies were carried out at 1% oxygen for 16 hours. PX-478 inhibited the hypoxia-induced increase in HIF-1α protein with IC₅₀s (mean±S.E., n=3) for PC-3 human prostate cancer cells of 3.9±2.0 μM, for MCF-7 breast cancer cells of 4.0±2.0 μM and for HT-29 colon cancer cells of 17.4±5.0 μM (FIG. 2A). PC-3 cells, but not MCF-7 or HT-29 cells, had detectable levels of HIF-1α under normoxic conditions. PX-478 decreased HIF-1α levels in PC-3 cells under normoxic conditions with an IC₅₀ value of 2.5±1.2 M. PX-478 caused no change in HIF-1α mRNA or HIF-1β protein under normoxic or hypoxic conditions. When PX-478 was removed HIF-1α protein returned to pre-treatment levels within 4 hour, see FIG. 2B.

To test the effect of PX-478 on transactivation by HIF-1, MCF-7 and HT-29 cells were transiently transfected with constructs expressing luciferase under the control of multiple copies of the HRE from phosphoglycerate kinase (PGK) or an empty vector control. Results were normalized for transfection efficiency using Renilla luciferase which is constitutively expressed. PX-478 treatment significantly decreased HIF-1 transactivation in both cell lines, see FIG. 2C. The hypoxia induced expression of VEGF protein, a HIF-1 regulated gene, was also significantly decreased by PX-478 treatment for 16 hours under hypoxic conditions, see FIG. 2D (p=<0.01), in a dose-dependent manner. VEGF protein production under normoxic conditions was not inhibited by PX-478.

### EXAMPLE 2

### PX-478 Does not Affect the Nuclear Localization of the HIF-1α HIF-1 Heterodimer in Hypoxic Cells

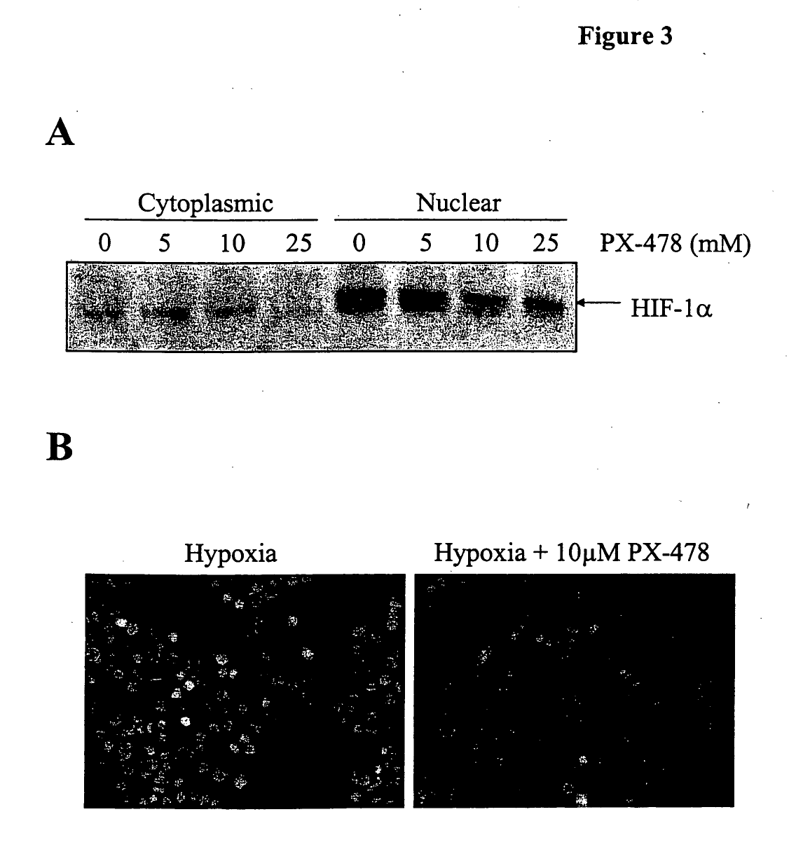

In FIG. 3, human breast cancer MCF-7, colon cancer HT-29, and prostate cancer PC-3, under hypoxic conditions were treated with PX-478. A. HT-29 cells were treated as indicated with PX-478 for 16 hour under normoxic (20% oxygen) or hypoxic (1% oxygen) conditions. HIF-1α levels were examined using Western blotting in nuclear and cytoplasmic fractions prepared from HT-29 cells treated with PX-478 under hypoxic conditions. PX-478 decreased HIF-1α levels in both subcellular fractions suggesting that PX-478 does not affect the nuclear import of the HIF-1α HIF-1 heterodimer in hypoxic cells. Nuclear and cytoplasmic extracts were prepared and HIF-1α levels were examined. B. HT-29 cells were treated for 16 hours under hypoxic conditions with or without 25M PX-478. Cells were fixed and permeabilized and HIF-1α protein was detected using immunofluorescence staining, confirmed that PX-478 does not affect the cellular localization of HIF-1α since no difference in subcellular localization of HIF-1α was observed after treatment although overall HIF-1α was decreased. Similar results were obtained in MCF-7 cells (data not shown).

### EXAMPLE 3

### Inhibition of HIF-1α is Associated with Increased HIF-1αDegradation and Ubiquitination

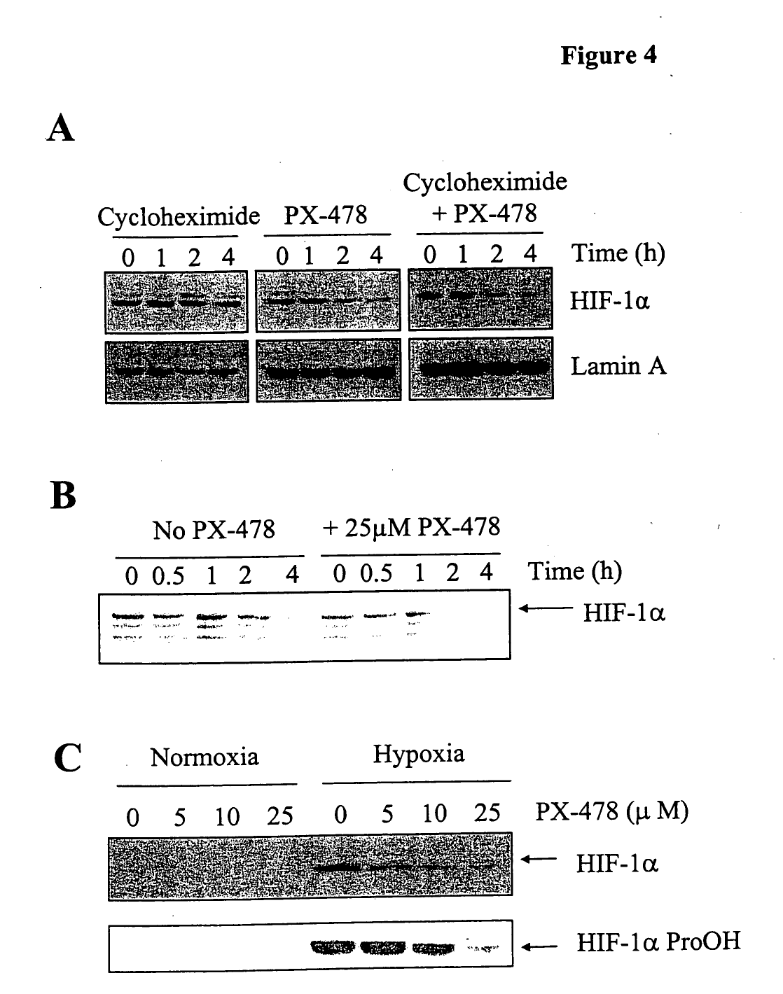

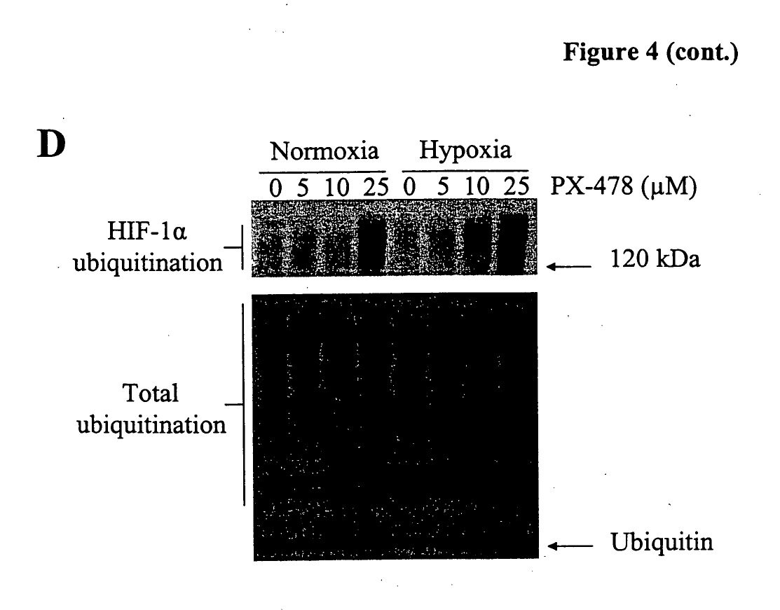

In FIG. 4, to determine whether PX-478 affects the breakdown of HIF-1α, PX-478 was co-incubated with the protein synthesis inhibitor cycloheximide during hypoxia treatment of HT-29 cells. A. HT-29 cells were treated with the protein synthesis inhibitor cycloheximide (40 M), PX-478 (25 M), or both, in hypoxia (1% oxygen). At the times indicated, nuclear cell extracts were prepared and HIF-1α levels were examined using Western blotting. Lamin A was used as a loading control. B. HT-29 cells were pulse-labeled with [³⁵S]-cysteine/methionine and chased in unlabeled medium for the indicated times. For PX-478-treated samples, PX-478 was added to the chase medium. C. HT-29 cells were exposed to PX-478 as indicated under normoxia (20% oxygen) or hypoxia (1% oxygen). Nuclear cell extracts were prepared, and HIF-1α and hydroxylated HIF-1α levels were examined using Western blotting. D. HT-29 cells were exposed to normoxia (20% oxygen) or hypoxia for 16 hours with PX-478 as indicated. Total cell extracts were prepared and ubiquitination of HIF-1α was determined using Western blotting after immunoprecipitation of HIF-1α (top panel). Total ubiquitination was measured in total cell extracts (pre-immunoprecipitation) using Western blotting (lower panel).

The results of FIG. 4 show that HIF-1α was stable for at least 4 h in the hypoxia and cycloheximide treated cells, while PX-478 treatment increased the rate of HIF-1α breakdown (FIG. 4A). Pulse chase experiments using [³⁵S]-methionine/cysteine showed that PX-478 decreases the half-life of HIF-1α from 3 h under hypoxic conditions to about 1.5 h in the presence of PX-478 (FIG. 4B). Similar results were obtained in MCF-7 cells (data not shown). PX-478 did not increase HIF-1α proline hydroxylation relative to total HIF-1α levels (FIG. 4C).

Examination of ubiquitination of HIF-1α after treatment with PX-478 in seen in FIG. 4D. PX-478 increased ubiquitination of HIF-1α in a dose-dependent manner under both normoxic and hypoxic conditions without affecting total cell protein ubiquitination (FIG. 4D). There was a greater increase in ubiquitination of HIF-1α by PX-478 with hypoxic than normoxic conditions. This was expected since HIF-1α levels are higher under hypoxic conditions and more HIF-1α will be available for immunoprecipitation. In addition to examining ubiquitination in total cell lysates, we also examined ubiquitination in the detergent-insoluble fraction, normally removed during preparation of cellular lysates. PX-478 did not increase levels of HIF-1α ubiquitination in this fraction (data not shown).

### EXAMPLE 4

### Inhibition of HIF-1α Occurs Independently of pVHL

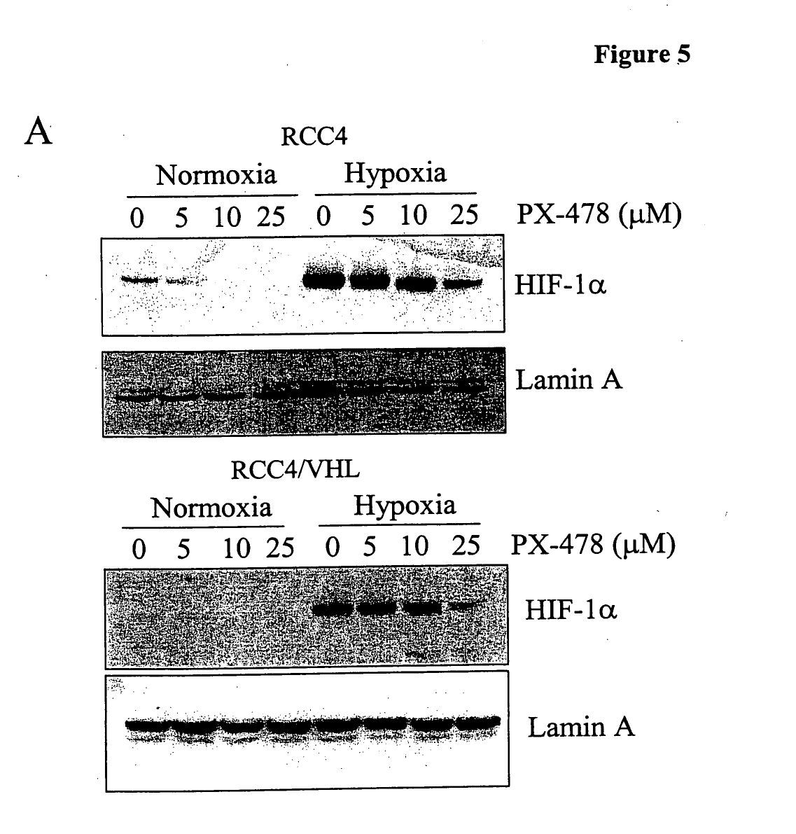

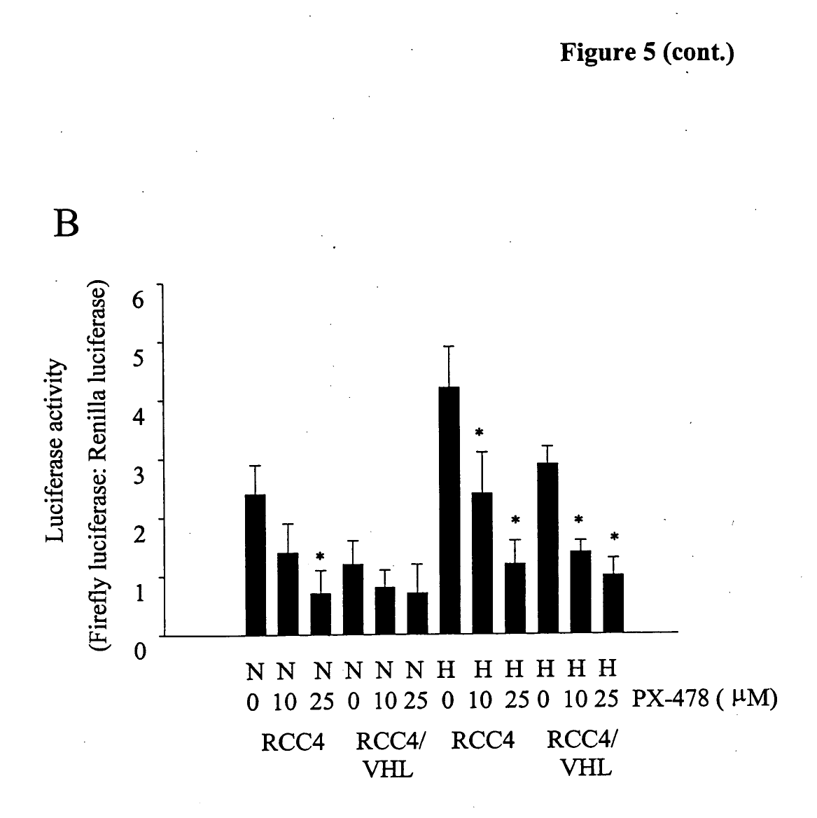

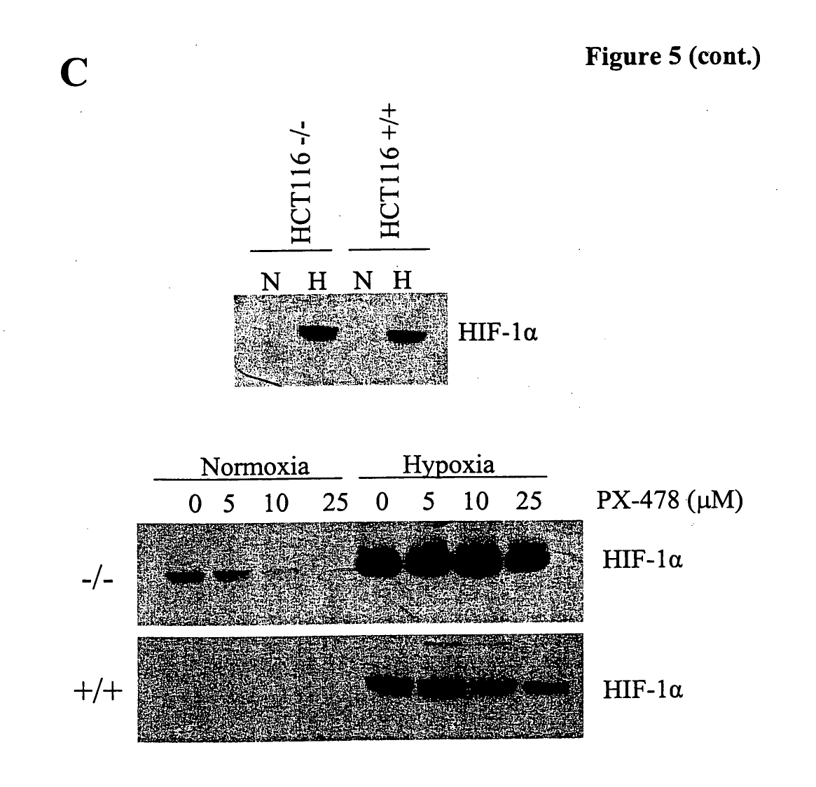

In FIG. 5, the pVHL pathway is tested. To test whether PX-478 decreases HIF-1α protein levels through the pVHL pathway, human renal cell carcinoma RCC4 cells lacking functional pVHL and the corresponding cell line (RCC4/VHL) containing functional pVHL through transfection of wild type pVHL were used. A. Human renal cancer RCC4 cells lacking pVHL (top panel) and RCC4/VHL cells into which pVHL has been re-transfected (lower panel) were exposed to PX-478 as indicated under normoxia (20% oxygen) or hypoxia (1% oxygen). RCC4 cells lacking pVHL and RCC4 cells into which pVHL has been re-transfected were obtained according to the methods disclosed in Maxwell P., Wiesener M., Chang G-W et al., The Tumor Suppressor Portein VHL targets hypoxia-inducible factors for oxygen-dependent proteolysis, Nature 1999; 399: 271-275. Nuclear cell extracts were prepared and HIF-1α levels were examined using Western blotting. Lamin A was used as a loading control. C. RCC4 and RCC4/VHL cells were transiently transfected with vectors expressing Firefly luciferase under the control of multiple copies of the HRE from PGK or constitutively expressing Renilla luciferase to control for transfection efficiency. Cells were exposed to normoxia or hypoxia for 16 hours in the presence of PX-478 as indicated. Luciferase activity was expressed as the ratio of Firefly: renilla luciferase. C. Human colon cancer HCT116+/+ cells expressing wild type p53 and HCT116−/− cells lacking p53 were exposed to normoxia or hypoxia (top panel) and also to doses of PX-478 as indicated under normoxia (20% oxygen) or hypoxia (1% oxygen) (lower panel). Nuclear cell extracts were prepared and HIF-1α levels were examined using Western blotting. Lamin A was used as a loading control.

RCC4 cells showed elevated levels of HIF-1α protein under normoxic conditions compared to RCC4/VHL cells, as expected, and hypoxia further increased HIF-1α protein in both cell lines (FIG. 5A). The additional increase in both HIF-1α protein and HIF-1 transactivation under hypoxia compared to normoxia (FIG. 5A and B) demonstrate that HIF-1α may also be regulated by pVHL-independent mechanisms in these cells PX-478 decreased HIF-α levels in both RCC4 and RCC4/VHL cells under hypoxic conditions with IC₅₀ values of 6.9±1.9 μM and 13.5±1.3 μM, respectively, and 5.1±2.0 μM in RCC4 cells under normoxic conditions. HIF-1 transactivation by both RCC4 and RCC4/VHL cells may be significantly inhibited by treatment with PX-478 (p=<0.01) (FIG. 5B).

### EXAMPLE 5

### Inhibition of HIF-1α Occurs Independently of p53

Wild type tumor suppressor p53 binds to HIF-1α allowing recruitment of MDM2, an E3 ubiquitin-ligase, resulting in the degradation of both p53 and HIF-1α. Human colon carcinoma HCT116+/+ cells expressing wild type p53 and HCT116−/− cells from which p53 has been deleted by homologous recombination were used to investigate the effect of p53 on hypoxia-induced HIF-1α accumulation. HCT116−/− cells from which p53 has been deleted by homologous recombination may be obtained by the methods described in Bunz F., Dutriaux A., Lengauer C., Requirement for p53 and p21 to sustain G2 arrest after DNA damage, Science 1998; 282: 1497-1501, herein incorporated by reference. HIF-1α protein levels were increased under both normoxic and hypoxic conditions in HCT116−/− cells compared to HCT116+/+ cells (FIG. 5C, top panel). PX-478 decreased HIF-1α protein in both cell lines demonstrating that it is not acting by a p53 dependent mechanism (FIG. 5C, lower panel).

### EXAMPLE 6

### Inhibition of HIF-1α Occurs Independently of HSP-90

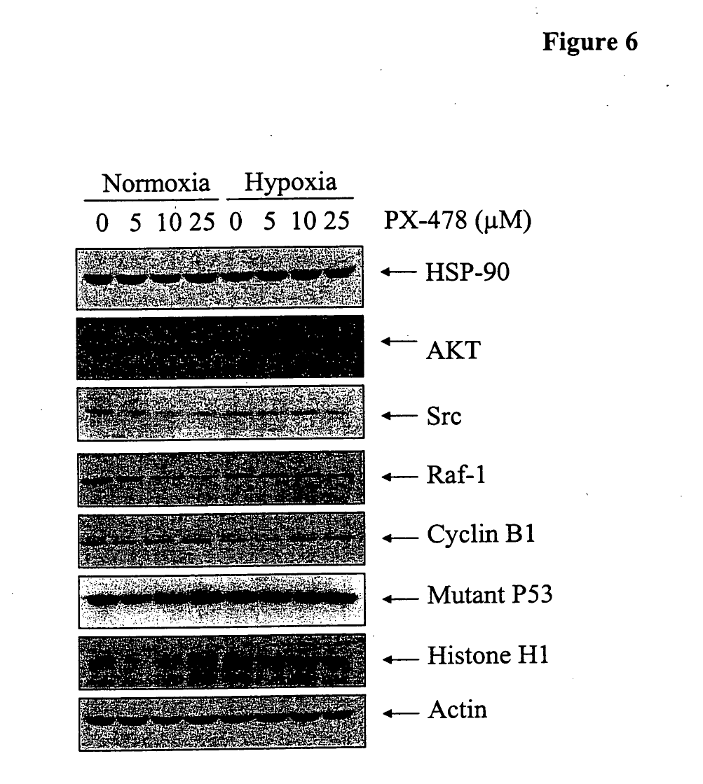

In FIG. 6, HT-29 cells were exposed to doses of PX-478 as indicated under normoxia (20% oxygen) or hypoxia (1% oxygen). Total cell extracts were prepared and levels of the proteins indicated were examined using Western blotting. The heat shock protein-90 (HSP-90), a molecular chaperone, has been previously implicated in HIF-1α degradation since the HSP-90 inhibitors geldanamycin and 17-allyl-amino desmethoxygeldanamycin (17-AAG) have been shown to inhibit both the basal and the hypoxia-induced increase in HIF-1α protein, independently of both pVHL and MDM2. See for example, Isaacs J. S., Jung Y. J., Mimnaugh E. G., Martinez A., Cuttita F., Neckers L. M., “Hsp90 Regulates a von hippel landau-independent hypoxia-inducible factore-1α-degradtive pathway,” J. Biol. Chem. 2002; 277:29936-29944, herein incorporated by reference.

To investigate whether PX-478 also acts through the HSP-90 mechanism, Western blotting was used to show that PX-478 treatment does not affect the levels of HSP-90 itself, or levels of the HSP-90 client proteins: AKT, c-Src and Raf-1. The ability of PX-478 to decrease the levels of other proteins whose degradation are known to be controlled by ubiquitination was also tested. PX-478 had no effect on the levels of cyclin B1, histone H1 or mutant p53 (FIG. 6).

### EXAMPLE 7

### Similar Mechanisms are Used in Vitro and in Vivo.

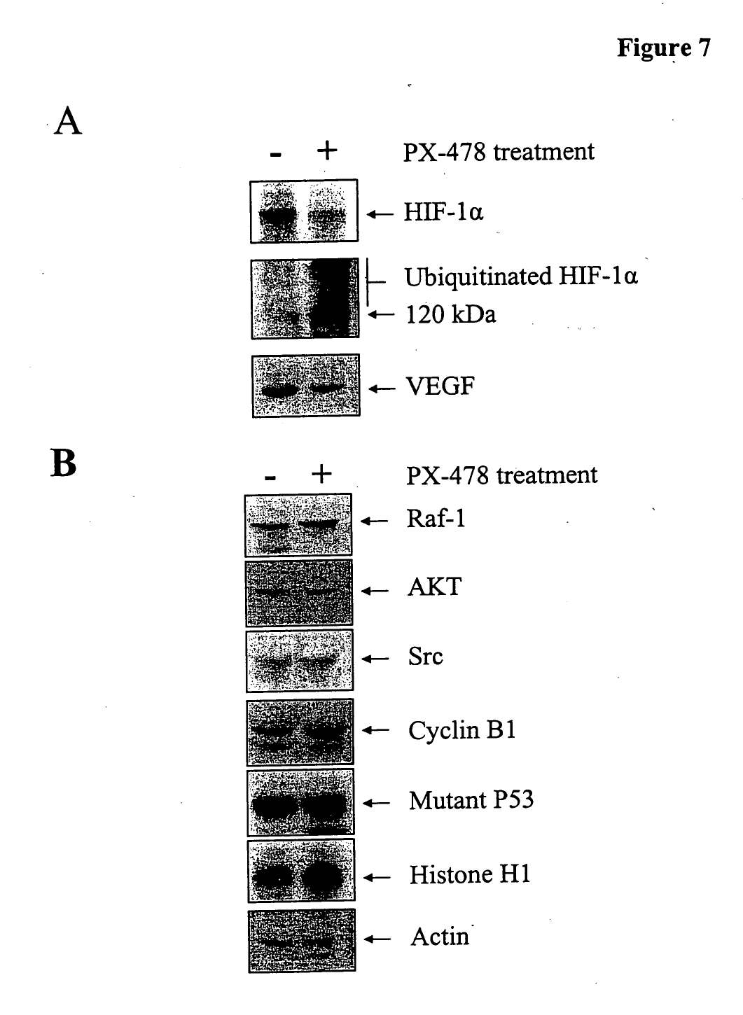

To demonstrate that results obtained in vitro were also seen in vivo, HT-29 colon tumor xenografts were grown s.c. in the flanks of scid mice. Mice were treated with 0 (−) or 100 (+) mg/kg PX-478. After two hours, tumors were harvested, homogenized in lysis buffer and total cell extracts were prepared. In FIG. 7 A, levels of HIF-1α and VEGF were measured using Western blotting and levels of ubiquitinated HIF-1α were measured by Western blotting after immunoprecipitation of HIF-1α. In FIG. 7B, levels of the proteins indicated were measured by Western blotting. This dose of PX-478 had been show to cause significant growth delay of HT-29 xenografts. See for example, Welsh S. J., Williams R. R., Kirkpatrick D. L., Powis G., “PX-478, a potent inhibitor of hypoxia-inducible factor-1 (HIF-1) and antitumor agent,” Eur. J. Cancer 2002: 38: 294. In FIG. 7A, Western blotting showed that PX-478 treatment caused a 60% decrease in HIF-1α protein, a 30% decrease in VEGF protein (FIG. 7A) and markedly increased ubiquitination of HIF-1α (FIG. 7A). No changes in AKT, Raf-1, Src-1, histone H1, cyclin B1 or mutant p53 were observed in the HT-29 tumors confirming the lack of effect of PX-478 on these proteins seen in vitro (FIG. 7B). Similar results of a decrease in HIF-1α protein levels and an increase in HIF-1α ubiquitination were obtained using PC-3 xenografts (data not shown).

### EXAMPLE 8

### Inhibition of Deubiquitination Regulates HIF-1α Levels

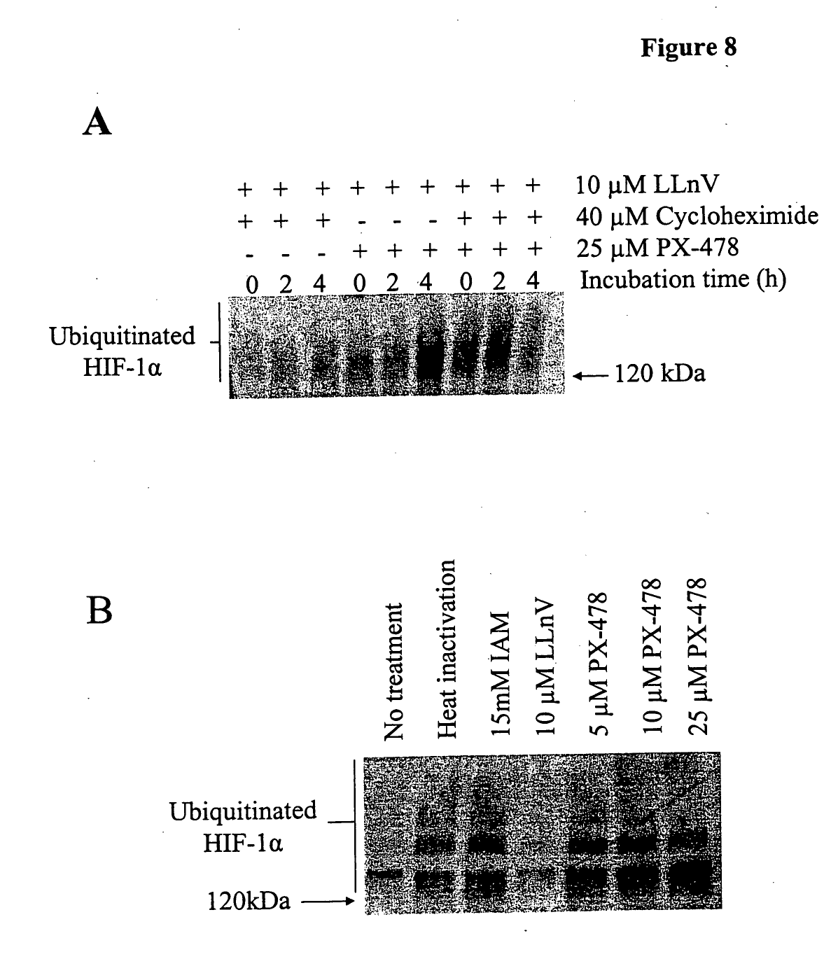

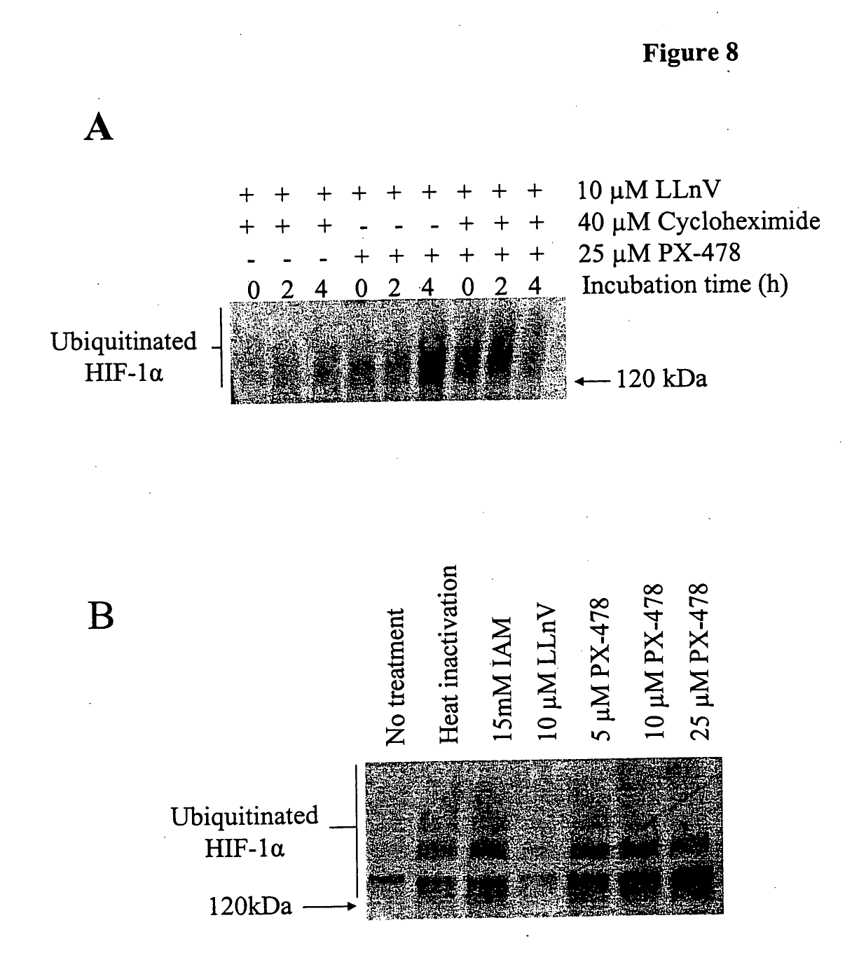

In FIG. 8A, HT-29 cells were treated for 16 hours in hypoxia (1% oxygen) in the presence of 10 M of the proteasome inhibitor LLnV. Cycloheximide (40 M) or PX-478 (25 M), as indicated, were added and cells were incubated in hypoxia for the times shown. Total cell extracts were prepared and levels of ubiquitinated HIF-1α was examined by Western blotting after immunoprecipitation of HIF-1α. In FIG. 8B, total cell extracts from HT-29 cells were incubated for 15 minutes in vitro with PX-478 at the doses indicated, 15 mM iodoacetamide (IAM), or 10 M LLnV. Heat was used to inactivate enzyme activity. Treated extracts were then incubated with polyubiquitinated HIF-1α. The deubiquitination of HIF-1α was followed using Western blotting for ubiquitin.

Polyubiquitination is a prerequisite step for degradation of most proteins by the 26S proteasome. In addition to ubiquitinating enzymes that link ubiquitin to proteins, there is a family of deubiquitinase enzymes that are responsible for removing ubiquitin moieties from ubiquitinated proteins allowing further regulation of the breakdown process. To further investigate how PX-478 affects ubiquitination, HT-29 cells were treated with PX-478 under hypoxic conditions in the presence of the proteasome inhibitor LLnV to prevent breakdown of HIF-1α. Cycloheximide alone had no affect on ubiquitination of HIF-1α (FIG. 8A). Treatment with PX-478 in the absence or presence of cycloheximide promoted a shift towards highly ubiquitinated species of HIF-1α compared to those detected without PX-478 treatment (FIG. 8A). The decrease in ubiquitinated HIF-1α at 25 M PX-478 with LLnV and cycloheximide is probably a result of the toxicity of the drug combination to the cells. Similar results were observed in MCF-7 cells (data not shown).

To determine if PX-478 inhibits deubiquitination specifically, an in vitro assay was performed using polyubiquitinated HIF-1α as a substrate and HT-29 cell lysate as the source of deubiquitinase enzyme activity. Ubiquitinated HIF-1α was detected using Western blotting for ubiquitin (FIG. 8B). Heat inactivation and treatment of lysate with 15 mM iodoacetamide prevented deubiquitination (FIG. 8B). Iodoacetamide has been shown to prevent deubiquitination. See, e.g., Strayhorm W. B., Wadziniski, B. E., “A Novel in Vitro Assay for Deubiquitination of I kappa B alpha,” Arch. Biochem. Biophys. 2002; 400:76-84. LLnV did not affect HIF-1α deubiquitination (FIG. 8B). Incubation with untreated lysate resulted in the disappearance of all highly ubiquitinated species of HIF-1α. PX-478 at even 5 μM, the lowest concentration tested, prevented the deubiquitination of HIF-1α (FIG. 8B). A dose-dependency was not observed with PX-478 added to lysates since it is likely to be more potent than when added to cells. Similar results were obtained using MCF-7 and PC-3 cells (data not shown).

The preceding examples have shown that PX-478 suppresses HIF-1α protein levels in cancer cells with constitutive expression of HIF-1α under aerobic conditions (PC-3 prostate cancer and RCC4 renal cancer) and other cancer cells that only show increased HIF-1α under hypoxic conditions (MCF-7 breast cancer, HT-29 colon cancer and HCT-116 colon cancer). The preceding examples have shown that the decrease in HIF-1α caused by PX-478 is associated with an increase in levels of ubiquitinated HIF-1α. The preceding examples have shown that the PX-478 regulates HIF-1α through the ubuitin/26S proteasome pathway.

While preferred embodiments have been described in detail, variations may be made to these embodiments without departing from the spirit or scope of the attached claims.

1. A therapeutic compound for the regulation of HIF-1α levels in cells under normoxic or hypoxic conditions comprising PX-478.

2. A pharmaceutical formulation comprising the compound of claim 1, together with a pharmaceutically acceptable carrier or diluent.

3. The pharmaceutical formulation of claim 2, wherein the acceptable carrier may be selected from the group consisting of water for injection, buffered aqueous solutions, and powdered salts.

4. A method of regulating levels of HIF-1α in cells under normoxic or hypoxic conditions comprising administering to a patient PX-478.

5. The method of claim 4, wherein cellular protein levels of HSP-90 client proteins, cyclin B1, mutant p53 and histone H1 are not substantially affected by the administration of PX-478.

6. The method of claim 4, wherein PX-478 is administered locally, orally or systemically.

7. The method of claim 4, wherein PX-478 is administered in a pharmaceutical formulation together with a pharmaceutically acceptable carrier or diluent.

8. The method of claim 7, wherein the acceptable carrier may be selected from the group consisting of water for injection, buffered aqueous solutions, and powdered salts.

9. The method of claim 4, wherein PX-478 is administered in a dosage of about 0.001 mg per kg to about 1000 mg per kg body weight of the patient.

10. A method of decreasing HIF-1α protein levels in cells under normoxic or hypoxic conditions comprising administering to a patient PX-478.

11. The method of claim 10, wherein cellular protein levels of HSP-90 client proteins, cyclin B1, mutant p53 and histone H1 are not substantially affected by the administration of PX-478.

12. The method of claim 10, wherein PX-478 is administered locally, orally or sytemically.

13. The method of claim 10, wherein PX-478 is administered in a pharmaceutical formulation together with a pharmaceutically acceptable carrier or diluent.

14. The method of claim 13, wherein the acceptable carrier may be selected from the group consisting of water for injection, buffered aqueous solutions, and powdered salts.

15. The method of claim 10, wherein PX-478 is administered in a dosage of about 0.001 mg per kg to about 1000 mg per kg body weight of the patient.

16. A method of decreasing HIF-1 transactivation activity in cells under normoxic or hypoxic conditions comprising administering to a patient PX-478.

17. The method of claim 16, wherein cellular protein levels of HSP-90 client proteins, cyclin B1, mutant p53 and histone H1 are not substantially affected by the administration of PX-478.

18. The method of claim 16, wherein PX-478 is administered locally, orally or systemically.

19. The method of claim 16, wherein PX-478 is administered in a pharmaceutical formulation together with a pharmaceutically acceptable carrier or diluent.

20. The method of claim 19, wherein the acceptable carrier may be selected from the group consisting of water for injection, buffered aqueous solutions, and powdered salts.

21. The method of claim 16, wherein PX-478 is administered in a dosage of about 0.001 mg per kg to about 1000 mg per kg body weight of the patient.

22. A method of regulating HIF-1α degradation by the 26S proteasome in cells under normoxic or hypoxic conditions comprising administering to a patient PX-478.

23. The method of claim 22, wherein cellular protein levels of HSP-90 client proteins, cyclin B1, mutant p53 and histone H1 are not substantially affected by the administration of PX-478.

24. The method of claim 22, wherein PX-478 is administered locally, orally or systemically.

25. The method of claim 22, wherein PX-478 is administered in a pharmaceutical formulation together with a pharmaceutically acceptable carrier or diluent.

26. The method of claim 25, wherein the acceptable carrier may be selected from the group consisting of water for injection, buffered aqueous solutions, and powdered salts.

27. The method of claim 22, wherein PX-478 is administered in a dosage of about 0.001 mg per kg to about 1000 mg per kg body weight of the patient.

28. A method of increasing ubiquitination of HIF-1α in cells under normoxic or hypoxic conditions comprising administering to a patient PX-478.

29. The method of claim 28, wherein cellular protein levels of HSP-90 client proteins, cyclin B1, mutant p53 and histone H1 are not substantially affected by the administration of PX-478.

30. The method of claim 28, wherein PX-478 is administered locally, orally or systemically.

31. The method of claim 28, wherein PX-478 is administered in a pharmaceutical formulation together with a pharmaceutically acceptable carrier or diluent.

32. The method of claim 31, wherein the acceptable carrier may be selected from the group consisting of water for injection, buffered aqueous solutions, and powdered salts.

33. The method of claim 28, wherein PX-478 is administered in a dosage of about 0.001 mg per kg to about 1000 mg per kg body weight of the patient.

34. A method of inhibiting the deubiquitination of HIF-1α in cells under normoxic or hypoxic conditions comprising administering to a patient PX-478.

35. The method of claim 34, wherein cellular protein levels of HSP-90 client proteins, cyclin B1, mutant p53 and histone H1 are not substantially affected by the administration of PX-478.

36. The method of claim 34, wherein PX-478 is administered locally, orally or systemically.

37. The method of claim 34, wherein PX-478 is administered in a pharmaceutical formulation together with a pharmaceutically acceptable carrier or diluent.

38. The method of claim 37, wherein the acceptable carrier may be selected from the group consisting of water for injection, buffered aqueous solutions, and powdered salts.

39. The method of claim 34, wherein PX-478 is administered in a dosage of about 0.001 mg per kg to about 1000 mg per kg body weight of the patient.
